(19) United States
(12) Patent Application Publication
(10) Pub. No.: US 2016/0318891 A1
(43) Pub. Date: Nov. 3, 2016

(54) TONABERSAT PRODRUGS

(71) Applicant: PROXIMAGEN LIMITED, Cambridge (GB)

(72) Inventors: Edward Savory, Cambourne (GB); Martyn Pritchard, St. Ives (GB); Michael Higginbottom, Caldecote (GB); William John O'Neil, Camelford (GB)

(73) Assignee: PROXIMAGEN LIMITED, Cambridge (GB)

(21) Appl. No.: 15/107,578

(22) PCT Filed: Dec. 22, 2014
(86) PCT No.: PCT/GB2014/053819

§ 371 Date: Jun. 23, 2016

(30) Foreign Application Priority Data
Dec. 23, 2013 (GB) 1322932.3

(51) Int. Cl. C07D 311/68; C07F 9/655; C07D 405/12

(52) U.S. Cl. CPC C07D 311/68; C07D 405/12; C07F 9/65522

(57) ABSTRACT

The invention relates to pharmaceutically active compounds having improved pharmacokinetic properties and being useful for the treatment or prevention of a range of conditions including migraine, epilepsy, non-epileptic seizures, brain injury (including stroke, intracranial haemorrhage and trauma induced) or cardiovascular diseases including myocardial infarction, coronary revascularization or angina. The compounds of the invention form a novel group of related prodrugs of formula (II), where Ar is a 3-chloro-4-fluorophenyl ring, a 3-chlorophenyl ring or a 4-fluorophenyl ring; and R is a hydrolysable group comprising an amino group or an acidic group.

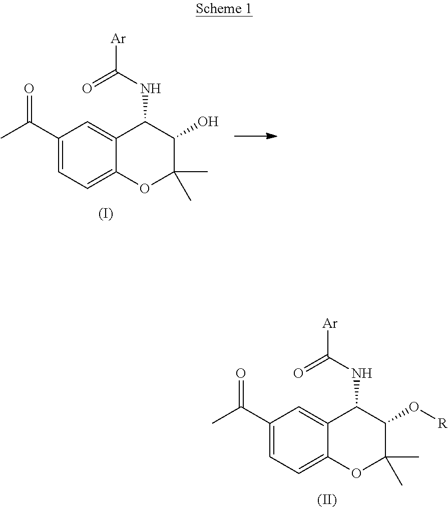

The present invention relates to pharmaceutically active compounds having improved pharmacokinetic properties, the compounds being useful for the treatment or prevention of a range of conditions including migraine, epilepsy, non-epileptic seizures, brain injury (including stroke, intracranial haemorrhage and trauma induced) or cardiovascular disease including myocardial infarction, coronary revascularization or angina.

### BACKGROUND TO THE INVENTION

Cortical spreading depolarization (CSD) is a wave of depolarisation with consequent depressed electrical activity which spreads across the surface of the cerebral cortex (at a rate of 2-6 mm/min) usually followed by hyperaemia and neuronal hyperpolarisation. The reduction in electrical activity is a consequence of neuron depolarisation and swelling, with K+ efflux, Na and Ca influx and electrical silence. This abnormal neuronal activity is associated with delayed neuronal damage in a number of pathological states including cerebral ischaemia (arising from e.g. stroke, haemorrhage and traumatic brain injury Strong et al., 2002 Fabricius et al., 2006; Dreier et al., 2006 Dohmen et al., 2008), epilepsy and the aura associated with migraine (Lauritzen 1994; Goadsby 2007). As the CSD wave moves across the cortex it is associated with a reactive increase in local blood flow which may serve to help restore the more normal ionic balance of the neurons affected. After the CSD induced hyperaemia the local increase in blood flow attenuates (oligaemia) potentially resulting in imbalances in energy supply and demand. Under certain conditions, the reactive hyperaemia is not observed, but instead the local vasculature constricts resulting in ischaemia which in turn can lead to neuronal death. The conditions triggering this abnormal response in experimental models are high extracellular levels of K+ and low NO availability. These conditions are typically seen in ischaemic areas of the brain, and clusters of CSD waves in these circumstances result in spreading ischaemia (see Dreier 2011). Of particular importance is the spreading ischaemia seen after sub-arachnoid haemorrhage (SAH), in the penumbra of an infarct and after traumatic brain injury where delayed neuronal damage can have a significant effect on clinical outcomes (Dreier et al., 2006, 2012; Hartings et al., 2011a, 2011b; Fabricius et al., 2006).

Given the detrimental effect of clusters of CSDs in humans and experimental animals, and the poor prognosis associated with CSDs, there is an unmet medical need for new compounds useful for inhibiting CSDs for patients with and without brain injuries. Without wishing to be bound by theory, the spread of CSD is believed to be mediated by gap junctions rather than by neuronal synaptic communication (Nedergard et al., 1995; Rawanduzy et al., 1997, Saito et al., 1997), the gap junctions providing a means of spreading the depolarisation in the absence of normal synaptic communication. Gap junctions are comprised of connexin proteins of which there are 21 in the human genome. Each Gap junction is made of two hemichannels, each comprising six connexin monomers.

Gap junctions are also implicated in a number of other disease states including hereditary diseases of the skin and ear (e.g. keratitis-ichthyosis deafness syndrome, erythrokeratoderma variabilis, Vohwinkel's syndrome, and hypotrichosis-deafness syndrome). Blockade of gap junction proteins has been shown to beneficial in some preclinical models of pain (e.g. Spataro et al., 2004 J Pain 5, 392-405, Wu et al., 2012 J Neurosci Res. 90,337-45). This is believed to be a consequence of gap junction blockade in the spinal cord resulting in a reduction in the hypersensitivity of the dorsal horn to sensory nerve input. In addition gap junctions and their associated hemichannels have been implicated in neurodegenerative diseases including Alzheimer's disease, Parkinson's Disease, Huntington's Disease and amyotrophic lateral sclerosis (Takeuchi et al 2011 PLoS One.; 6, e21108).

Tonabersat (SB-220453/PRX201145) is a gap junction blocker (Silberstein, 2009; Durham and Garrett, 2009) which binds selectively and with high affinity to a unique stereo-selective site in rat and human brains. Consistent with its action on gap junctions Tonabersat also inhibits high K+ evoked CSD in cats (Smith et al., 2000; Read et al., 2000; Bradley et al., 2001) and rats (Read et al., 2001).

However, known gap junction blockers, including Tonabersat and Carabersat, suffer from undesirable physiochemical properties. Tonabersat is a crystalline solid with a high melting point (152-153C) and with a relatively high lipophilicity (log P 3.32). The compound has no readily ionisable groups and consequently has a low aqueous solubility of 0.025 mg/ml over a range of pH values including pH of 7.4. The low aqueous solubility of Tonabersat makes both intravenous (IV) and oral (PO) modes of administration problematic. The poor aqueous solubility prevents rapid injection of the required dose of Tonabersat which is required for the treatment of head injuries and stroke or for emergency treatment of epileptic seizures where the patient may be unconscious and unable to swallow an oral drug. At present the effective plasma concentrations needed to reduce the cortical spreading depression caused by head injury or stroke can only be reached by slow IV infusion given over a period of hours. With respect to the PO administration of Tonabersat for the treatment of other indications, solubility limited dissolution of the tablet form of Tonabersat given PO leads to a significant “food effect” with differences in the maximum blood concentration of Tonabersat (Cmax) seen depending on whether the drug is given with or without food. These differences make it difficult to accurately predict the plasma exposure of Tonabersat when given orally, thus increasing the risk of under or over dosing the patient.

Therefore it is an object of the present invention to provide gap junction blocker compounds having improved physiochemical properties thus improving the utility of these agents in treating a range of disease states.

### BRIEF DESCRIPTION OF THE INVENTION

The present invention makes available novel pro-drug compounds.

### DETAILED DESCRIPTION OF THE INVENTION

The present invention makes available a pro-drug compound selected from:
(3S,4S)-6-Acetyl-4-[(3-chloro-4-fluorobenzene)amido]-2,2-dimethyl-3,4-dihydro-2H-1-benzopyran-3-yl (2S)-2-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-4-methylpentanoate;
(3S,4S)-6-Acetyl-4-[(3-chloro-4-fluorobenzene)amido]-2,2-dimethyl-3,4-dihydro-2H-1-benzopyran-3-yl (2S)-pyrrolidine-2-carboxylate;
(3S,4S)-6-Acetyl-4-[(3-chloro-4-fluorobenzene)amido]-2,2-dimethyl-3,4-dihydro-2H-1-benzopyran-3-yl 2-amino-2-methylpropanoate;
(3S,4S)-6-Acetyl-4-[(3-chloro-4-fluorobenzene)amido]-2,2-dimethyl-3,4-dihydro-2H-1-benzopyran-3-yl 3-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2-(methylamino)propanoate;
(3S,4S)-6-Acetyl-4-[(3-chloro-4-fluorobenzene)amido]-2,2-dimethyl-3,4-dihydro-2H-1-benzopyran-3-yl (2S)-2,6-diaminohexanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-carbamoylpropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-4-carbamoylbutanoate;
(3S,4S)-6-Acetyl-4-[(3-chloro-4-fluorobenzene)amido]-2,2-dimethyl-3,4-dihydro-2H-1-benzopyran-3-yl (2S)-2-amino-5-carbamimidamidopentanoate;
(3S)-4-{[(3S,4S)-6-Acetyl-4-[(3-chloro-4-fluorobenzene)amido]-2,2-dimethyl-3,4-dihydro-2H-1-benzopyran-3-yl]oxy}-3-amino-4-oxobutanoic acid;
(4S)-5-{[(3S,4S)-6-Acetyl-4-[(3-chloro-4-fluorobenzene)amido]-2,2-dimethyl-3,4-dihydro-2H-1-benzopyran-3-yl]oxy}-4-amino-5-oxopentanoic acid;
({[(3S,4S)-6-Acetyl-4-[(3-chloro-4-fluorobenzene)amido]-2,2-dimethyl-3,4-dihydro-2H-1-benzopyran-3-yl]oxy}methoxy)(methoxy)phosphinic acid;
N-[(3S,4S)-6-Acetyl-3-{[(2S)-2,3-dihydroxypropoxy]methoxy}-2,2-dimethyl-3,4-dihydro-2H-1-benzopyran-4-yl]-3-chloro-4-fluorobenzamide;
{[(3S,4S)-6-Acetyl-4-[(4-fluorobenzene)amido]-2,2-dimethyl-3,4-dihydro-2H-1-benzopyran-3-yl]oxy}phosphonic acid;
({[(3S,4S)-6-acetyl-4-[(4-fluorobenzene)amido]-2,2-dimethyl-3,4-dihydro-2H-1-benzopyran-3-yl]oxy}methoxy)phosphonic acid;
{[(3S,4S)-6-Acetyl-4-[(3-chlorobenzene)amido]-2,2-dimethyl-3,4-dihydro-2H-1-benzopyran-3-yl]oxy}phosphonic acid; and
({[(3S,4S)-6-acetyl-4-[(3-chlorobenzene)amido]-2,2-dimethyl-3,4-dihydro-2H-1-benzopyran-3-yl]oxy}methoxy)phosphonic acid
   or a hydrate, solvate, or pharmaceutically acceptable salt thereof.

In an embodiment, the compounds of the invention form a novel group of related prodrugs of formula (II), where Ar is a 3-chloro,4-fluorophenyl ring, or a 3-chlorophenyl ring, or a 4-fluorophenyl ring; and R is a hydrolysable group.

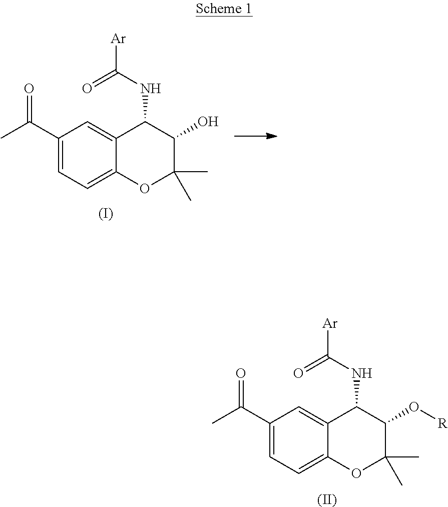

In an embodiment, the compounds of the invention have in common a hydrolysable group comprising an amino group, and those compounds are:
(3S,4S)-6-Acetyl-4-[(3-chloro-4-fluorobenzene)amido]-2,2-dimethyl-3,4-dihydro-2H-1-benzopyran-3-yl (2S)-2-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-4-methylpentanoate;
(3S,4S)-6-Acetyl-4-[(3-chloro-4-fluorobenzene)amido]-2,2-dimethyl-3,4-dihydro-2H-1-benzopyran-3-yl (2S)-pyrrolidine-2-carboxylate;
(3S,4S)-6-Acetyl-4-[(3-chloro-4-fluorobenzene)amido]-2,2-dimethyl-3,4-dihydro-2H-1-benzopyran-3-yl 2-amino-2-methylpropanoate;
(3S,4S)-6-Acetyl-4-[(3-chloro-4-fluorobenzene)amido]-2,2-dimethyl-3,4-dihydro-2H-1-benzopyran-3-yl 3-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2-(methylamino)propanoate;
(3S,4S)-6-Acetyl-4-[(3-chloro-4-fluorobenzene)amido]-2,2-dimethyl-3,4-dihydro-2H-1-benzopyran-3-yl (2S)-2,6-diaminohexanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-carbamoylpropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-4-carbamoylbutanoate;
(3S,4S)-6-Acetyl-4-[(3-chloro-4-fluorobenzene)amido]-2,2-dimethyl-3,4-dihydro-2H-1-benzopyran-3-yl (2S)-2-amino-5-carbamimidamidopentanoate;
(3S)-4-{[(3S,4S)-6-Acetyl-4-[(3-chloro-4-fluorobenzene)amido]-2,2-dimethyl-3,4-dihydro-2H-1-benzopyran-3-yl]oxy}-3-amino-4-oxobutanoic acid;
   or a hydrate, solvate, or pharmaceutically acceptable salt thereof.

In an embodiment, the compounds of the invention have in common a hydrolysable group derived from a natural alpha-amino acid, and those compounds are:
(3S,4S)-6-Acetyl-4-[(3-chloro-4-fluorobenzene)amido]-2,2-dimethyl-3,4-dihydro-2H-1-benzopyran-3-yl (2S)-2-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-4-methylpentanoate;
(3S,4S)-6-Acetyl-4-[(3-chloro-4-fluorobenzene)amido]-2,2-dimethyl-3,4-dihydro-2H-1-benzopyran-3-yl (2S)-pyrrolidine-2-carboxylate;
(3S,4S)-6-Acetyl-4-[(3-chloro-4-fluorobenzene)amido]-2,2-dimethyl-3,4-dihydro-2H-1-benzopyran-3-yl (2S)-2,6-diaminohexanoate;
(3S,4S)-6-Acetyl-4-[(3-chloro-4-fluorobenzene)amido]-2,2-dimethyl-3,4-dihydro-2H-1-benzopyran-3-yl (2S)-2-amino-5-carbamimidamidopentanoate;
(3S)-4-{[(3S,4S)-6-Acetyl-4-[(3-chloro-4-fluorobenzene)amido]-2,2-dimethyl-3,4-dihydro-2H-1-benzopyran-3-yl]oxy}-3-amino-4-oxobutanoic acid;
(4S)-5-{[(3S,4S)-6-Acetyl-4-[(3-chloro-4-fluorobenzene)amido]-2,2-dimethyl-3,4-dihydro-2H-1-benzopyran-3-yl]oxy}-4-amino-5-oxopentanoic acid;
   or a hydrate, solvate, or pharmaceutically acceptable salt thereof.

In an embodiment, the compounds of the invention have in common a hydrolysable group comprising an acidic group, and those compounds are:
({[(3S,4S)-6-Acetyl-4-[(3-chloro-4-fluorobenzene)amido]-2,2-dimethyl-3,4-dihydro-2H-1-benzopyran-3-yl]oxy}methoxy)(methoxy)phosphinic acid
{[(3S,4S)-6-Acetyl-4-[(4-fluorobenzene)amido]-2,2-dimethyl-3,4-dihydro-2H-1-benzopyran-3-yl]oxy}phosphonic acid ({[(3S,4S)-6-acetyl-4-[(4-fluorobenzene)amido]-2,2-dimethyl-3,4-dihydro-2H-1-benzopyran-3-yl]oxy}methoxy)phosphonic acid;
{[(3S,4S)-6-Acetyl-4-[(3-chlorobenzene)amido]-2,2-dimethyl-3,4-dihydro-2H-1-benzopyran-3-yl]oxy}phosphonic acid; and
({[(3S,4S)-6-acetyl-4-[(3-chlorobenzene)amido]-2,2-dimethyl-3,4-dihydro-2H-1-benzopyran-3-yl]oxy}methoxy)phosphonic acid
   or a hydrate, solvate, or pharmaceutically acceptable salt thereof.

### TERMINOLOGY

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds of the invention which are basic can form salts, including pharmaceutically acceptable salts with inorganic acids, e.g. hydrohalic acids such as hydrochloric or hydrobromic acids, sulphuric acid, nitric acid or phosphoric acid and the like, and with organic acids e.g. acetic, tartaric, succinic, fumaric, maleic, malic, salicylic, citric, methanesulphonic, p-toluenesulphonic, benzoic, benzenesunfonic, glutamic, lactic, and mandelic acids and the like.

The formation of specific salt forms can provide compounds of the invention with improved physicochemical properties. For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomers with R or S stereochemistry at each chiral axis. The invention includes all such enantiomers and diastereoisomers and mixtures thereof.

The present invention makes available a pharmaceutical composition comprising a compound as claimed in claim 1 together with one or more pharmaceutically acceptable carriers and/or excipients.

The present invention makes available a compound of a compound as claimed in claim 1 for use in medicine.

In an embodiment, the invention encompasses the use of a compound a compound as claimed in claim 1 for treatment of a disease or medical condition which benefits from inhibition of gap junction activity. Inhibition of gap junction activity may be achieved by blocking the gap junction as a whole or by blocking one or more hemichannels.

In an embodiment, the invention encompasses a method of treatment of a disease or medical condition which benefits from inhibition of gap junction activity, comprising administering to a subject suffering from such disease or condition and effective amount of a compound of a compound as claimed in claim 1

In an embodiment the disease or condition which benefits from inhibition of gap junction activity is selected from among migraine, aura with or without migraine, epilepsy, non-epileptic seizures, cerebrovascular accidents including stroke, intracranial haemorrhage (including traumatic brain injury, epidural hematoma, subdural hematoma and subarachnoid haemorrhage), and intra-cerebral haemorrhage, spinal cord vascular accidents arising from trauma, epidural hematoma, subdural hematoma or subarachnoid haemorrhage, pain including pain arising from hyperalgesia caused by damage to sensory neurons (i.e. neuropathic pain including but not limited to diabetic neuropathy, polyneuropathy, cancer pain, fibromyalgia, myofascial pain, post herpetic neuralgia, spinal stenosis, HIV pain, post-operative pain, post-trauma pain) or inflammation (including pain associated with osteoarthritis, rheumatoid arthritis, sciatica/radiculopathy, pancreatitis, tendonitis), neurodegenerative disease (including but not limited to Alzheimer's Disease, Parkinson's Disease, Huntington's Disease and Amyotrophic Lateral Sclerosis) and cardiovascular disease including myocardial infarction, coronary revascularization or angina.

It will be understood that the pharmacology of the brain is a complex and constantly evolving area of research. Without wishing to be bound by theory, it is currently hypothesised that the claimed compounds exert their therapeutic effect by inhibiting gap junction activity. However, it is anticipated that the claimed compounds may exert their therapeutic effect by additional and/or alternative mechanisms of action. For the avoidance of doubt, the claimed compounds are expected to be useful for treatment of any one of the diseases selected form among migraine, aura with or without migraine, epilepsy, non-epileptic seizures, cerebrovascular accidents including stroke, intracranial haemorrhage (including traumatic brain injury, epidural hematoma, subdural hematoma and subarachnoid haemorrhage), and intra-cerebral haemorrhage, spinal cord vascular accidents arising from trauma, epidural hematoma, subdural hematoma or subarachnoid haemorrhage, pain including pain arising from hyperalgesia caused by damage to sensory neurons (i.e. neuropathic pain including but not limited to diabetic neuropathy, polyneuropathy, cancer pain, fibromyalgia, myofascial pain, post herpetic neuralgia, spinal stenosis, HIV pain, post-operative pain, post-trauma pain) or inflammation (including pain associated with osteoarthritis, rheumatoid arthritis, sciatica/radiculopathy, pancreatitis, tendonitis), neurodegenerative disease (including but not limited to Alzheimer's Disease, Parkinson's Disease, Huntington's Disease and Amyotrophic Lateral Sclerosis) and cardiovascular disease including myocardial infarction, coronary revascularization or angina.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. However, for administration to human patients, the total daily dose of the compounds of the invention may typically be in the range 1 mg to 1000 mg depending, of course, on the mode of administration. For example, oral administration may require a total daily dose of from 10 mg to 1000 mg, while an intravenous dose may only require from 1 mg to 500 mg. The total daily dose may be administered in single or divided doses and may, at the physician's discretion, fall outside of the typical range given herein. These dosages are based on an average human subject having a weight of about 60 kg to 100 kg. The physician will readily be able to determine doses for subjects whose weight falls outside this range, such as infants and the elderly, and especially obese patients.

The compounds with which the invention is concerned may be prepared for administration by any route consistent with their pharmacokinetic properties. Suitable routes for administration include oral, intravenous, buccal, intranasal, inhalation, rectal, and intradermal. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

The pro-drug may also be administered parenterally in a sterile medium. Depending on the vehicle and concentration used, the drug can either be suspended or dissolved in the vehicle. Advantageously, adjuvants such as local anaesthetic, preservative and buffering agents can be dissolved in the vehicle. The person skilled in the art is aware of many excipients useful for IV formulation.

### PREPARATION OF COMPOUNDS OF THE INVENTION

The following abbreviations have been used:
Ala Alanine
aq Aqueous
Boc tertiary-butyloxycarbonyl
d day(s)
calcd calculated
DCC N,N′-Dicyclohexylcarbodiimide
DCM dichloromethane
DMAP 4-dimethylaminopyridine
DME dimethyl ether
DMF dimethylformamide
ES+, ESI+ electrospray ionization
EtOAc ethyl acetate
Et₂O diethyl ether
Et₃N triethylamine
h hour(s)
HATU (O-(7-azabenzotriazol-1-yl)-N,N,N′,N′-tetramethyluronium hexafluorophosphate)
HPLC High Performance Liquid Chromatography
HRMS High-Resolution Mass Spectrometry
Int Intermediate
LCMS Liquid Chromatography Mass Spectrometry
M molar
MeCN Acetonitrile
MEK Methylethyl ketone
MeOH methanol
MTBE methyl tertiary-butyl ether
[MH]⁺/[MH]⁻ protonated/deprotonated molecular ion
MS Mass Spectrometry
NIS N-iodosuccinimide
NMM N-methylmorpholine
Rt retention time
sat saturated
THF tetrahydrofuran
Val Valine

### EXAMPLES AND INTERMEDIATE COMPOUNDS

### Experimental Methods

Reactions were conducted at room temperature unless otherwise specified. Preparative chromatography was performed using a Flash Master Personal system equipped with Isolute Flash II silica columns or using a CombiFlash Companion system equipped with GraceResolv silica column, unless otherwise stated. The purest fractions were collected, concentrated and dried under vacuum. Compounds were typically dried in a vacuum oven at 40° C. prior to purity analysis. Compound analysis was performed by HPLC/LCMS using an Agilent 1100 HPLC system/Waters ZQ mass spectrometer connected to an Agilent 1100 HPLC system with a Phenomenex Synergi, RP-Hydro column (150×4.6 mm, 4 μm, 1.5 mL per min, 30° C., gradient 5-100% MeCN (+0.085% TFA) in water (+0.1% TFA) over 7 min, 200-300 nm). The compounds prepared were named using IUPAC nomenclature. Accurate masses were measured using a Waters QTOF electrospray ion source and corrected using Leucine Enkephalin lockmass. Spectra were acquired in positive and negative electrospray mode. The acquired mass range was m/z 100-1000. Samples were dissolved in DMSO to give 1 mg/mL solutions which were then further diluted with Acetonitrile (50%)/Water (50%) to 1 μg/mL solutions prior to analysis. The values reported correspond either to the protonated or deprotonated molecular ions [MH]⁺ or [MH]⁻.

### Intermediate 1

### N-[(3S,4S)-6-Acetyl-2,2-dimethyl-3-[(methylsulfanyl)methoxy]-3,4-dihydro-2H-1-benzopyran-4-yl]-3-chloro-4-fluorobenzamide

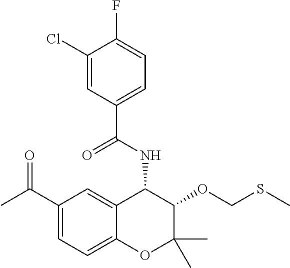 [SMILES: CSCO[C@H]1[C@@H](NC(=O)c2ccc(F)c(Cl)c2)c2cc(C(C)=O)ccc2OC1(C)C]

N-[(3S,4S)-6-Acetyl-3-hydroxy-2,2-dimethyl-3,4-dihydro-2H-1-benzopyran-4-yl]-3-chloro-4-fluorobenzamide (1.00 g, 2.55 mmol) was dissolved in DME (10 mL) and added to a suspension of sodium hydride (60% dispersion in oil, 112 mg, 2.81 mmol) in DME (10 mL). The reaction mixture was stirred for 10 min and NaI (421 mg, 2.81 mmol) and chloromethyl methyl sulfide (232 μL, 2.81 mmol) were added. The reaction mixture was stirred for 18 h, quenched with sat aq (NH₄)₂CO₃ solution (5 mL), diluted with EtOAc (50 mL), washed with water (3×25 mL), dried (MgSO₄) and concentrated in vacuo. The residue was purified by column chromatography to give the title compound (405 mg, 35%) as a white solid. LCMS (ES⁺): 452.1 [MH]⁺. HPLC: Rt 6.36 min, 86.2% purity.

### Intermediate 2

### ({[(3S,4S)-6-Acetyl-4-[(3-chloro-4-fluorobenzene)amido]-2,2-dimethyl-3,4-dihydro-2H-1-benzopyran-3-yl]oxy}methoxy)phosphonic acid diamine

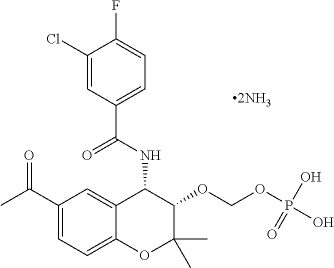 [SMILES: CC(=O)c1ccc2c(c1)[C@H](NC(=O)c1ccc(F)c(Cl)c1)[C@H](OCOP(=O)(O)O)C(C)(C)O2]

Intermediate 1 (600 mg, 1.33 mmol) was dissolved in THF (12 mL) and 85% aq phosphoric acid (911 mg, 9.29 mmol) and powdered 4 Å molecular sieves (1.80 g) were added. The reaction mixture was cooled to 0° C. and NIS (478 mg, 2.12 mmol) was added. The reaction mixture was stirred overnight, diluted with EtOAc (30 mL), filtered and washed with 5% aq Na₂S₂O₃ (25 mL). The organic fraction was extracted with 10% aq Na₂CO₃ (30 mL). The aqueous fraction was acidified to pH1-2 with 2M HCl in ice and extracted with EtOAc (15 mL). The organic fraction was washed with water (3×10 mL) and concentrated in vacuo. The residue was partitioned between EtOAc (20 mL) and 5% aq Na₂CO₃ (20 mL). The aqueous fraction was acidified to pH 1 with 2M HCl in ice and purified by column chromatography (Amberlite XAD-4 resin (5 g), eluent water/MeCN) and trituration from EtOAc/MTBE. The residue was partitioned between water (10 mL) and EtOAc (5 mL), 7M ammonia in MeOH (1 mL) was added and the aqueous fraction was separated and concentrated in vacuo. The residue was triturated from EtOAc to give the title compound (44.0 mg, 6%) as a white solid. HPLC: Rt 4.88 min, 98.5% purity.

### Intermediate 3

### N-[(3S,4S)-6-Acetyl-3-hydroxy-2,2-dimethyl-3,4-dihydro-2H-1-benzopyran-4-yl]-4-fluorobenzamide

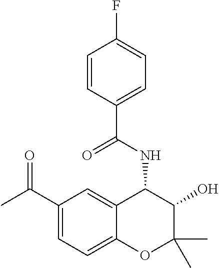 [SMILES: CC(=O)c1ccc2c(c1)[C@H](NC(=O)c1ccc(F)cc1)[C@H](O)C(C)(C)O2]

4-Fluorobenzoyl chloride (1.99 g, 12.6 mmol) was dissolved in DCM (5 mL) and added drop-wise to a solution of 1-[(3S,4S)-4-amino-3-hydroxy-2,2-dimethyl-3,4-dihydro-2H-1-benzopyran-6-yl]ethan-1-one sulfuric acid hydrate (4.00 g, 11.4 mmol) and Et₃N (5.57 mL) in DCM (70 mL) at 5° C. The reaction mixture was warmed to room temperature over 3 h and concentrated in vacuo. The residue was partitioned between EtOAc and water and the aqueous fraction was extracted with EtOAc. The combined organic fractions were washed with 1M aq HCl, 10% aq NaHCO₃ and brine, dried (MgSO₄) and concentrated in vacuo. The residue was crystallised from EtOAc/hexane to give the title compound (3.40 g, 83%). LCMS (ES⁺): 358.1 [MH]⁺.

### Intermediate 4

### N-[(3S,4S)-6-Acetyl-3-hydroxy-2,2-dimethyl-3,4-dihydro-2H-1-benzopyran-4-yl]-3-chlorobenzamide

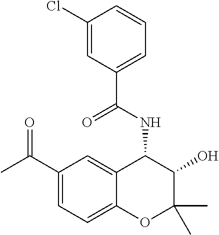 [SMILES: CC(=O)c1ccc2c(c1)[C@H](NC(=O)c1cccc(Cl)c1)[C@H](O)C(C)(C)O2]

Intermediate 4 was prepared similarly to Intermediate 3, using 3-chlorobenzoyl chloride instead of 4-fluorobenzoyl chloride, to give the crude title compound (4.38 g). LCMS (ES⁺): 374.1 [MH]⁺.

### Example 1

### (3S,4S)-6-Acetyl-4-[(3-chloro-4-fluorobenzene)amido]-2,2-dimethyl-3,4-dihydro-2H-1-benzopyran-3-yl (2S)-2-aminopropanoate hydrochloride

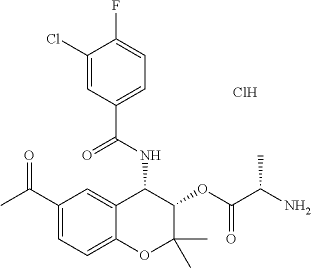 [SMILES: CC(=O)c1ccc2c(c1)[C@H](NC(=O)c1ccc(F)c(Cl)c1)[C@H](OC(=O)[C@H](C)N)C(C)(C)O2 ; Cl]

N-[(3S,4S)-6-Acetyl-3-hydroxy-2,2-dimethyl-3,4-dihydro-2H-1-benzopyran-4-yl]-3-chloro-4-fluorobenzamide (5.00 g, 12.8 mmol), Boc-Ala-OH (3.38 g, 17.9 mmol) and DMAP (160 mg, 1.31 mmol) were dissolved in DCM (150 mL) and a solution of DCC (3.95 g, 19.1 mmol) in DCM (20 mL) was added drop-wise at 0° C. The reaction mixture was stirred for 3 h, filtered through Celite and concentrated in vacuo. The residue was purified by column chromatography and triturated from hexane. The resulting Boc intermediate (7.15 g) was dissolved in MeOH (10 mL), a solution of 4M HCl in dioxane (100 mL) was added and the reaction mixture was stirred for 3.5 h. The reaction mixture was concentrated in vacuo and the residue was triturated from hexane/Et₂O (1:1) to give the crude title compound (6.30 g). LCMS (ES⁺): 463.1 [MH]⁺. A portion of the residue was purified by column chromatography (eluting with 100:2.5:0.5 DCM/MeOH/NH₄OH), addition of 2M HCl in Et₂O and concentration in vacuo to give the title compound as a yellow solid (81.3 mg). HPLC: Rt 5.09 min, 97.4% purity; HRMS (ESI+) calcd for C23H24ClFN2O5 463.144. found 463.144.

### Examples 2-14

Examples 2-14 were prepared similarly to Example 1, using the appropriate Boc-protected amino acid; see Table 1 below.

TABLE 1
Amide formation and Boc-deprotection

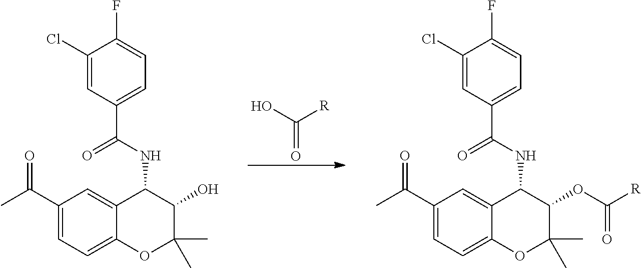

Where RCO₂H is an appropriate Boc-protected amino acid

| | | | Form, |
|---|---|---|---|
| Ex | Structure | Name | Yield, Analytical data |
| 2 | 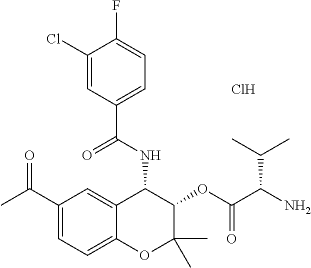 [SMILES: CC(=O)c1ccc2c(c1)[C@H](NC(=O)c1ccc(F)c(Cl)c1)[C@H](OC(=O)[C@@H](N)C(C)C)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl (2S)-2-amino-3-methylbutanoate hydrochloride | White solid. 565 mg, 54% HPLC: Rt 5.38 min, 98.1%. HRMS (ESI+) calcd for C₂₅H₂₈ClFN₂O₅ 491.175 found 491.173 |
| 3 | 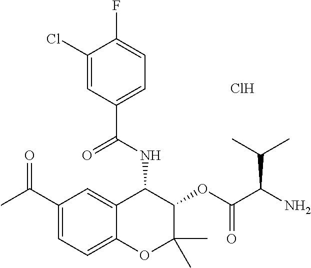 [SMILES: CC(=O)c1ccc2c(c1)[C@H](NC(=O)c1ccc(F)c(Cl)c1)[C@H](OC(=O)[C@H](N)C(C)C)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl (2R)-2-amino-3-methylbutanoate hydrochloride | Pale yellow solid. 417 mg, 25% HPLC: Rt 5.33 min, 100%. HRMS (ESI+) calcd for C₂₅H₂₈ClFN₂O₅ 491.175 found 491.177 |
| 4 | 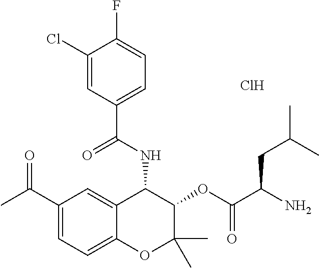 [SMILES: CC(=O)c1ccc2c(c1)[C@H](NC(=O)c1ccc(F)c(Cl)c1)[C@H](OC(=O)[C@H](N)CC(C)C)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl (2R)-2-amino-4-methylpentanoate hydrochloride | Beige solid. 387 mg, 36% HPLC: Rt 5.57 min, 98.3%. HRMS (ESI+) calcd for C₂₆H₃₀ClFN₂O₅ 505.191 found 505.193 |
| 5 | 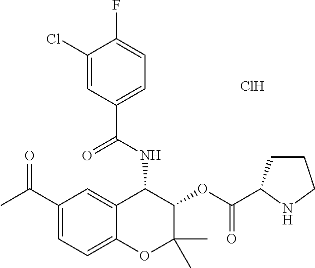 [SMILES: CC(=O)c1ccc2c(c1)[C@H](NC(=O)c1ccc(F)c(Cl)c1)[C@H](OC(=O)[C@@H]1CCCN1)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl (2S)-pyrrolidine-2-carboxylate hydrochloride | Cream solid. 210 mg, 11% HPLC: Rt 5.25 min, 96.0%. HRMS (ESI+) calcd for C₂₅H₂₆ClFN₂O₅ 489.159 found 489.158 |
| 6 | 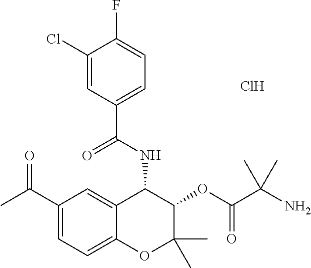 [SMILES: CC(=O)c1ccc2c(c1)[C@H](NC(=O)c1ccc(F)c(Cl)c1)[C@H](OC(=O)C(C)(C)N)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl 2- amino-2-methylpropanoate hydrochloride | Beige solid. 121 mg, 6% HPLC: Rt 5.23 min, 97.7%. HRMS (ESI+) calcd for C₂₄H₂₆ClFN₂O₅ 477.159 found 477.158 |
| 7 | 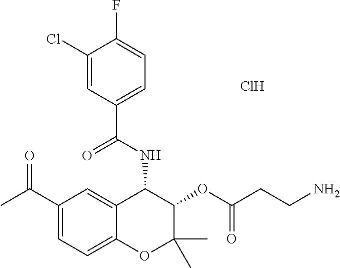 [SMILES: CC(=O)c1ccc2c(c1)[C@H](NC(=O)c1ccc(F)c(Cl)c1)[C@H](OC(=O)CCN)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl 3- aminopropanoate hydrochloride | White solid. 460 mg, 60% HPLC: Rt 5.06 min, 99.2%. HRMS (ESI+) calcd for C₂₃H₂₄ClFN₂O₅ 463.1436 found 463.1437 |
| 8 | 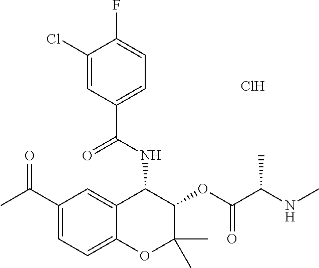 [SMILES: CN[C@@H](C)C(=O)O[C@H]1[C@@H](NC(=O)c2ccc(F)c(Cl)c2)c2cc(C(C)=O)ccc2OC1(C)C ; Cl] | (3S,4S)-6-Acetyl-4-[(3-chloro-4- fluorobenzene)amido]-2,2-dimethyl- 3,4-dihydro-2H-1-benzopyran-3-yl (2S)-2-(methylamino)propanoate hydrochloride | White solid. 300 mg, 67% HPLC: Rt 5.18 min, 99.0%. HRMS (ESI+) calcd for C₂₄H₂₆ClFN₂O₅ 477.159 found 477.159 |
| 9 | 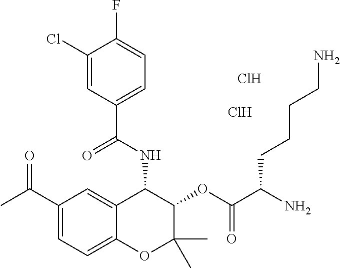 [SMILES: CC(=O)c1ccc2c(c1)[C@H](NC(=O)c1ccc(F)c(Cl)c1)[C@H](OC(=O)[C@@H](N)CCCCN)C(C)(C)O2 ; Cl ; Cl] | (3S,4S)-6-Acetyl-4-[(3-chloro-4- fluorobenzene)amido]-2,2-dimethyl- 3,4-dihydro-2H-1-benzopyran-3-yl (2S)-2,6-diaminohexanoate dihydrochloride | Pale pink solid. 629 mg, 62% HPLC: Rt 4.48 min, 95.3%. HRMS (ESI+) calcd for C₂₆H₃₁ClFN₃O₅ 520.201 found 520.203 |
| 10 | 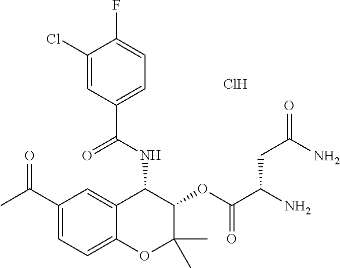 [SMILES: CC(=O)c1ccc2c(c1)[C@H](NC(=O)c1ccc(F)c(Cl)c1)[C@H](OC(=O)[C@@H](N)CC(N)=O)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl (2S)-2-amino-3-carbamoylpropanoate hydrochloride | White solid. 425 mg, 39% HPLC: Rt 4.83 min, 96.5%. HRMS (ESI+) calcd for C₂₄H₂₅ClFN₃O₆ 506.149 found 506.149 |
| 11 | 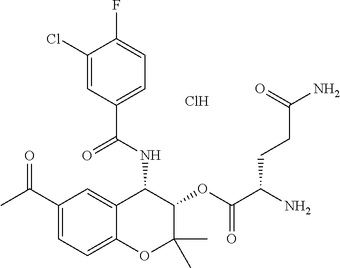 [SMILES: CC(=O)c1ccc2c(c1)[C@H](NC(=O)c1ccc(F)c(Cl)c1)[C@H](OC(=O)[C@@H](N)CCC(N)=O)C(C)(C)O2 ; Cl] | (3S,4S)-6-Acetyl-4-[(3-chloro-4- fluorobenzene)amido]-2,2-dimethyl- 3,4-dihydro-2H-1-benzopyran-3-yl (2S)-2-amino-4-carbamoylbutanoate hydrochloride | Off white solid. 424 mg, 75% HPLC: Rt 4.85 min, 93.1%. HRMS (ESI+) calcd for C₂₅H₂₇ClFN₃O₆ 520.165 found 520.165 |
| 12 | 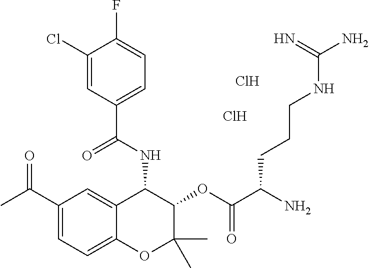 [SMILES: CC(=O)c1ccc2c(c1)[C@H](NC(=O)c1ccc(F)c(Cl)c1)[C@H](OC(=O)[C@@H](N)CCCNC(=N)N)C(C)(C)O2 ; Cl ; Cl] | (3S,4S)-6-Acetyl-4-[(3-chloro-4- fluorobenzene)amido]-2,2-dimethyl- 3,4-dihydro-2H-1-benzopyran-3-yl (2S)-2-amino-5- carbamimidamidopentanoate dihydrochloride | Pink solid. 76 mg, 24% HPLC: Rt 4.54 min, 98.5%. HRMS (ESI+) calcd for C₂₆H₃₁ClFN₅O₅ 548.208 found 548.207 |
| 13 | 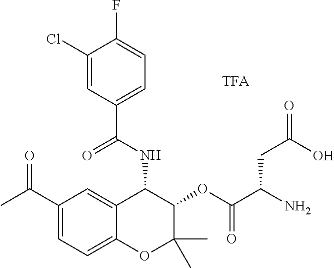 [SMILES: CC(=O)c1ccc2c(c1)[C@H](NC(=O)c1ccc(F)c(Cl)c1)[C@H](OC(=O)[C@@H](N)CC(=O)O)C(C)(C)O2 ; "TFA"] | (3S)-4-{[(3S,4S)-6-Acetyl-4-[(3-chloro- 4-fluorobenzene)amido]-2,2-dimethyl- 3,4-dihydro-2H-1-benzopyran-3- yl]oxy}-3-amino-4-oxobutanoic acid; trifluoroacetic acid | White solid. 86 mg, 37% HPLC: Rt 5.04 min, 99.3%. HRMS (ESI+) calcd for C₂₄H₂₄ClFN₂O₇ 507.133 found 507.134 |
| 14 | 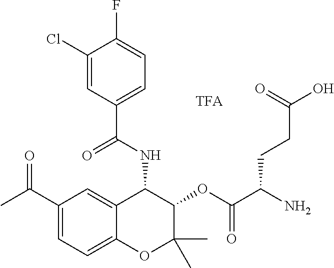 [SMILES: CC(=O)c1ccc2c(c1)[C@H](NC(=O)c1ccc(F)c(Cl)c1)[C@H](OC(=O)[C@@H](N)CCC(=O)O)C(C)(C)O2 ; "TFA"] | (4S)-5-{[(3S,4S)-6-Acetyl-4-[(3-chloro- 4-fluorobenzene)amido]-2,2-dimethyl- 3,4-dihydro-2H-1-benzopyran-3- yl]oxy}-4-amino-5-oxopentanoic acid; trifluoroacetic acid | White solid. 360 mg, 45% HPLC: Rt 5.07 min, 98.2%. HRMS (ESI+) calcd for C₂₅H₂₆ClFN₂O₇ 521.150 found 521.150 |

### Example 15

### ({[(3S,4S)-6-Acetyl-4-[(3-chloro-4-fluorobenzene)amido]-2,2-dimethyl-3,4-dihydro-2H-1-benzopyran-3-yl]oxy}methoxy)(methoxy)phosphinic acid

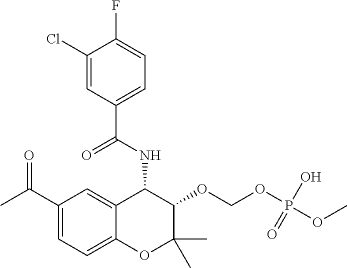 [SMILES: COP(=O)(O)OCO[C@H]1[C@@H](NC(=O)c2ccc(F)c(Cl)c2)c2cc(C(C)=O)ccc2OC1(C)C]

Intermediate 2 (500 mg, free acid, 1.00 mmol) was dissolved in MeOH (86 mL), DMAP (12.2 mg, 0.10 mmol) and DCC (440 mg, 2.13 mmol) were added and the reaction mixture was stirred overnight and concentrated in vacuo. The residue was partitioned between water (25 mL) and EtOAc (35 mL), filtered and the aqueous fraction was extracted with EtOAc (3×10 mL). The combined organic fractions were dried (MgSO₄) and concentrated in vacuo. The residue was purified by trituration from EtOAc/heptane and reverse phase column chromatography to give the title compound (61.6 mg, 12%) as a white solid. HPLC: Rt 5.03 min, 99.6% purity. HRMS (ESI+) calcd for C₂₂H₂₄ClFNO₈P 516.099. found 516.100.

### Example 16

### N-[(3S,4S)-6-Acetyl-3-{[(2S)-2,3-dihydroxypropoxy]methoxy}-2,2-dimethyl-3,4-dihydro-2H-1-benzopyran-4-yl]-3-chloro-4-fluorobenzamide

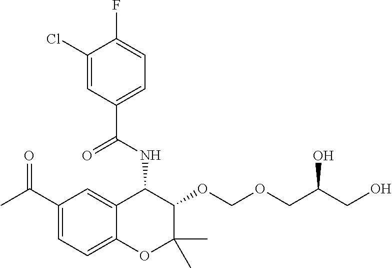 [SMILES: CC(=O)c1ccc2c(c1)[C@H](NC(=O)c1ccc(F)c(Cl)c1)[C@H](OCOC[C@@H](O)CO)C(C)(C)O2]

Intermediate 1 (1.00 g, 2.21 mmol) and R-(−)-2,2-dimethyl-1,3-dioxolane-4-methanol (1.65 mL, 13.0 mmol) were dissolved in 2-methyl-THF (30 mL), cooled to −30° C. and NIS (1.00 g, 4.00 mmol) and trifluoromethanesulfonic acid (4 drops) were added. The reaction mixture was stirred at −30° C. for 1 h, diluted with 1M aq sodium thiosulfate (50 mL) and extracted with EtOAc (4×50 mL). The combined organic fractions were washed with sat aq NaHCO₃ (100 mL), brine (100 mL), dried (MgSO₄) and concentrated in vacuo. The residue was dissolved in THF (30 mL), 2M aq HCl (30 mL) was added and the reaction mixture was stirred for 2 h, diluted with brine (100 mL) and extracted with EtOAc (4×50 mL). The combined organic fractions were dried (MgSO₄) and concentrated in vacuo. The residue was purified by column chromatography to give the title compound (420 mg, 38%) as a white solid. LCMS (ES⁺): 517.9 [MNa]⁺. HPLC: Rt 5.38 min, 98.6% purity.

### Example 17

### {[(3S,4S)-6-Acetyl-4-[(4-fluorobenzene)amido]-2,2-dimethyl-3,4-dihydro-2H-1-benzopyran-3-yl]oxy}phosphonic acid

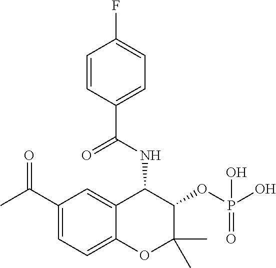 [SMILES: CC(=O)c1ccc2c(c1)[C@H](NC(=O)c1ccc(F)cc1)[C@H](OP(=O)(O)O)C(C)(C)O2]

Intermediate 3 (400 mg, 1.12 mmol) and pyridine (365 uL, 4.48 mmol) were dissolved in MEK (11 mL), POCl₃ (330 uL, 3.58 mmol) was added and the reaction mixture was stirred for 20 h. The reaction mixture was filtered and the precipitate was washed with MEK (10 mL). 2M aq HCl (2 mL) was added to the combined filtrates and the reaction mixture was stirred at 65° C. for 1 h. The organic fraction was washed with brine (2×5 mL) and concentrated in vacuo. The residue was triturated from EtOAc (12 mL) and washed with EtOAc. The residue was slurried in MeCN (×3) and collected by filtration to give the title compound (375 mg, 77%). HPLC: Rt 4.48 min, 98.6% purity. HRMS (ESI−) calcd for C₂₀H₂₁FNO₇P 436.0962. found 436.0974.

### Example 18

### Sodium {[(3S,4S)-6-acetyl-4-[(4-fluorobenzene)amido]-2,2-dimethyl-3,4-dihydro-2H-1-benzopyran-3-yl]oxy}methyl hydrogen phosphate

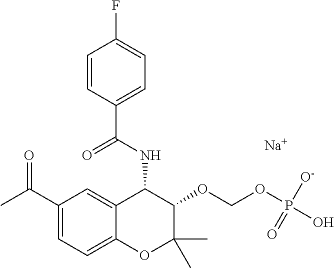 [SMILES: CC(=O)c1ccc2c(c1)[C@H](NC(=O)c1ccc(F)cc1)[C@H](OCOP(=O)([O-])O)C(C)(C)O2 ; [Na+]]

Example 18 was prepared similarly to Intermediate 2, using Intermediate 3 instead of N-[(3S,4S)-6-Acetyl-3-hydroxy-2,2-dimethyl-3,4-dihydro-2H-1-benzopyran-4-yl]-3-chloro-4-fluorobenzamide, to give the title compound (670 mg, 34%). HPLC: Rt 4.62 min, 99.7% purity. HRMS (ESI−) calcd for C₂₁H₂₂FNO₈P 466.1067. found 466.1053.

### Example 19

### {[(3S,4S)-6-Acetyl-4-[(3-chlorobenzene)amido]-2,2-dimethyl-3,4-dihydro-2H-1-benzopyran-3-yl]oxy}phosphonic acid

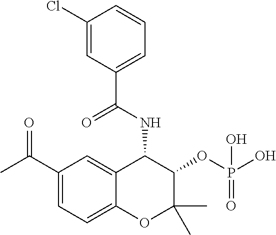 [SMILES: CC(=O)c1ccc2c(c1)[C@H](NC(=O)c1cccc(Cl)c1)[C@H](OP(=O)(O)O)C(C)(C)O2]

Example 19 was prepared similarly to Example 17, using Intermediate 4 instead of Intermediate 3, to give the title compound (264 mg, 54%). HPLC: Rt 4.78 min, 98.9% purity. HRMS (ESI−) calcd for C₂₀H₂₁ClNO₇P 452.0666. found 452.068.

### Example 20

### Sodium {[(3S,4S)-6-acetyl-4-[(3-chlorobenzene)amido]-2,2-dimethyl-3,4-dihydro-2H-1-benzopyran-3-yl]oxy}methyl hydrogen phosphate

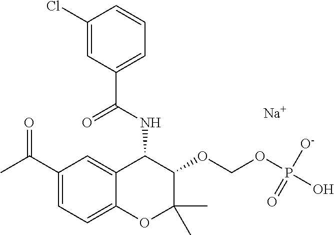 [SMILES: CC(=O)c1ccc2c(c1)[C@H](NC(=O)c1cccc(Cl)c1)[C@H](OCOP(=O)([O-])O)C(C)(C)O2 ; [Na+]]

Example 20 was prepared similarly to Example 18, using Intermediate 4 instead of Intermediate 3, to give the title compound (760 mg, 38%). HPLC: Rt 4.90 min, 98.2% purity. HRMS (ESI−) calcd for C₂₁H₂₂ClNO₈P 482.0772. found 482.0781.

### Biological Rational

Without wishing to be bound by theory, the general mode of action of the claimed pro-drugs is as follows. For IV administration the high solubility conferred by the solubilising pro-moiety to the parent Tonabersat-like drug is expected to allow a rapid bolus injection whereupon the pro-drug will be quickly cleaved by plasma esterases/phosphatases to reveal the parent drug. For PO administration the mode of action is either where the solubilising pro-drug is predominantly cleaved in the gut by esterases/phosphatases prior to absorption of the parent drug into the systemic circulation, or where the solubilising pro-drug is absorbed intact and then quickly cleaved by plasma esterases/phosphatases to reveal the parent drug.

### Solubility

In an embodiment prodrugs of the present invention are suitable for oral administration. The skilled person understands that the pH of the gastrointestinal tract changes along its length. For example, the stomach has a pH of around pH 1.5 and the GI tract after the stomach has a pH of around 5 to 7.5. For more detail see, for example, Measurement of gastrointestinal pH profiles in normal ambulant human subjects, Gut. 1988 August; 29(8): 1035-1041. Improved solubility is expected to result in improved absorption, and therefore improved oral bioavailability. Thus improved solubility at any pH value between around pH 1.5 to 8 is expected to improve oral bioavailability. Compounds of the invention were assessed for solubility in aqueous solutions having a pH of from 2 to 10. In an embodiment prodrugs of the invention have a solubility of >0.5 mg/mL in an aqueous solution having a pH of from 2 to 8. In an embodiment prodrugs have a solubility of >5.0 mg/mL, or >10.0 mg/mL, >100.0 mg/mL, or >200.0 mg/mL. In an embodiment the prodrugs have the aforementioned aqueous solubility at a pH within the range of from 4 to 8, or from 6 to 8.

In an embodiment prodrugs of the invention are administered intravenously. High prodrug solubility is advantageous in order to reduce the volume of solution administered to the patient, and to reduce the risk of damage to the circulatory system. Solubility of >10 mg/mL is preferred. Yet more preferred is solubility of >30 mg/mL or >100.0 mg/mL. Yet more preferred is solubility of >200.0 mg/mL. The solubility is measured in an aqueous solution having a pH of from 2 to 10, which pH range is advantageous for intravenous prodrug delivery. See, for example, A guide on intravenous drug compatibilities based on their pH, Nasser S C et al./Pharmacie Globale (IJCP) 2010, 5 (01)). In an embodiment the prodrugs of the claimed invention have solubility of >10 mg/mL in an aqueous solution having a pH of from 2 to 10. The solubility of certain Examples is shown in Table 2

TABLE 2
Solubility

| Example | Solubility | |
|---|---|---|
| 1 | >10 | mg/mL (pH 4.0) |
| 2 | ~5 | mg/mL (pH 4.4) |
| 3 | <1 | mg/mL (pH 4.8) |
| 4 | >10 | mg/mL (pH 4.2) |
| 5 | >10 | mg/mL (pH 4.4) |
| 6 | >10 | mg/mL (pH 4.7) |
| 8 | >10 | mg/mL (pH 5.1) |
| 9 | >10 | mg/mL (pH 4.4) |
| 10 | <1 | mg/mL(pH 4.4) |
| 11 | >2 | mg/mL (pH 4.6) |
| 12 | >10 | mg/mL (pH 4.4) |
| 13 | >2 | mg/mL (pH 6.5) |
| 14 | >2 | mg/mL (pH 4.6) |
| 16 | <1 | mg/mL (pH 3.0) |

### Pharmacokinetics

Example Prodrugs of the claimed invention were dosed either intravenously or orally to fasted male Sprague Dawley rats. The rats underwent surgery for jugular vein cannulation 48 h prior to dosing. Following dosing, 0.25 mL blood samples were taken via the cannulae at 0, 5, 10, 20, 30, 45, 60, 120, 240 & 360 min in EDTA coated tubes. Tubes were spun at 13,000 rpm for 4 min and 100 ul of supernatant taken immediately and stored at −80° C. prior to analysis. Plasma samples were analysed by LC-MS/MS following extraction by protein precipitation, and levels of parent prodrug and tonabersat were measured by MRM (Multiple Reaction Monitoring) analysis against an extracted calibration curve of plasma samples spiked with the Example prodrug and tonabersat.

The exposure of tonabersat in plasma following dosing of the prodrugs of the invention was compared directly to the exposure observed following dosing of an equimolar amount of tonabersat under analogous assay conditions (5.00 mg/kg oral dosing or 0.78 mg/kg intravenous dosing). In an embodiment prodrugs of the present invention have >10% exposure of tonabersat obtained following either oral or intravenous dosing of the prodrug to a human or animal subject, compared to the exposure obtained from dosing an equimolar amount of tonabersat itself. In an embodiment the exposure of tonabersat following dosing of the prodrugs is >20%, or >30%, or >40%, or >50%, or preferably >70% compared to the exposure obtained from dosing an equimolar amount of tonabersat itself.

Table 3 shows the exposure of tonabersat obtained following either oral or intravenous dosing of prodrug Examples, compared to the exposure obtained from dosing an equimolar amount of tonabersat itself.

TABLE 3
Pharmacokinetics

% exposure of
tonabersat after dosing the
prodrugs of the invention via:

| Example | Oral dosing (po) | Intravenous dosing (iv) |
|---|---|---|
| 2 | 34% | 35% |
| 3 | 47% | — |
| 4 | 112% | — |
| 5 | 39% | 18% |
| 6 | 14% | 6% |
| 8 | 58% | 42% |
| 9 | 26% | 7% |
| 10 | 36% | 17% |
| 11 | 23% | 27% |
| 12 | 17% | 12% |
| 13 | 31% | 20% |
| 14 | 47% | 31% |
| 15 | 17% | <5% |
| 16 | 111% | <5% |

Examples 17-20 have substituents on the phenyl ring which do not correspond to the substituent of tonabersat. In the same way as Examples 1-16 hydrolyse in vivo to generate tonabersat, it is expected that Examples 17-20 will hydrolyse in vivo to generate corresponding drug compounds.

hERG Assay

Compounds of the invention were tested for inhibition of the human ether a go-go related gene (hERG) K⁺ channel using IonWorks patch clamp electrophysiology. 8 Point concentration-response curves were generated on two occasions using 3-fold serial dilutions from the maximum assay concentration (33 uM). Electrophysiological recordings were made from a Chinese Hamster Lung cell line stably expressing the full length hERG channel. Single cell ion currents were measured in the perforated patch clamp configuration (100 ug/mL amphoterocin) at room temperature using an IonWorks Quattro instrument. The internal solution contained 140 mM KCl, 1 mM MgCl₂, 1 mM EGTA and 20 mM HEPES and was buffered to pH 7.3. The external solution contained 138 mM NaCl, 2.7 mM KCl, 0.9 mM CaCl₂, 0.5 mM MgCl₂, 8 mM Na₂HPO₄ and 1.5 mM KH₂PO₄, and was buffered to pH 7.3. Cells were clamped at a holding potential of 70 mV for 30 s and then stepped to +40 mV for is. This was followed by a hyperpolarising step of 1 s to 30 mV to evoke the hERG tail current. This sequence was repeated 5 times at a frequency of 0.25 Hz. Currents were measured from the tail step at the 5^{th} pulse, and referenced to the holding current. Compounds were incubated for 6-7 min prior to a second measurement of the hERG signal using an identical pulse train. A minimum of 17 cells were required for each pIC50 curve fit. A control compound (quinidine) was used. In an embodiment the compounds of the invention have a hERG IC50 of >11 uM.

TABLE 4
hERG

| | hERG IC50 or % |
|---|---|
| Example | inhibition @ 11 uM |
| 1 | 6.0 uM |
| 2 | 38% @ 11 uM |
| 3 | 4.3 uM |
| 5 | 1.4 uM |
| 9 | 0% @ 11 uM |
| 10 | 18% @ 11 uM |
| 12 | 9% @ 11 uM |
| 13 | 19% @ 11 uM |
| 14 | 8% @ 11 uM |

1. A compound or a hydrate, solvate, or pharmaceutically acceptable salt thereof, the compound selected from:
(3S,4S)-6-Acetyl-4-[(3-chloro-4-fluorobenzene)amido]-2,2-dimethyl-3,4-dihydro-2H-1-benzopyran-3-yl (2R)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-4-methylpentanoate;
(3S,4S)-6-Acetyl-4-[(3-chloro-4-fluorobenzene)amido]-2,2-dimethyl-3,4-dihydro-2H-1-benzopyran-3-yl (2S)-pyrrolidine-2-carboxylate;
(3S,4S)-6-Acetyl-4-[(3-chloro-4-fluorobenzene)amido]-2,2-dimethyl-3,4-dihydro-2H-1-benzopyran-3-yl 2-amino-2-methylpropanoate;
(3S,4S)-6-Acetyl-4-[(3-chloro-4-fluorobenzene)amido]-2,2-dimethyl-3,4-dihydro-2H-1-benzopyran-3-yl 3-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2,6-diaminohexanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-carbamoylpropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-4-carbamoylbutanoate;
(3S,4S)-6-Acetyl-4-[(3-chloro-4-fluorobenzene)amido]-2,2-dimethyl-3,4-dihydro-2H-1-benzopyran-3-yl (2S)-2-amino-5-carbamimidamidopentanoate;
(3S)-4-{[(3S,4S)-6-Acetyl-4-[(3-chloro-4-fluorobenzene)amido]-2,2-dimethyl-3,4-dihydro-2H-1-benzopyran-3-yl]oxy}-3-amino-4-oxobutanoic acid;
(4S)-5-{[(3S,4S)-6-Acetyl-4-[(3-chloro-4-fluorobenzene)amido]-2,2-dimethyl-3,4-dihydro-2H-1-benzopyran-3-yl]oxy}-4-amino-5-oxopentanoic acid;
({[(3S,4S)-6-Acetyl-4-[(3-chloro-4-fluorobenzene)amido]-2,2-dimethyl-3,4-dihydro-2H-1-benzopyran-3-yl]oxy}methoxy)(methoxy)phosphinic acid;
N-[(3S,4S)-6-Acetyl-3-{[(2S)-2,3-dihydroxypropoxy]methoxy}-2,2-dimethyl-3,4-dihydro-2H-1-benzopyran-4-yl]-3-chloro-4-fluorobenzamide;
{[(3S,4S)-6-Acetyl-4-[(4-fluorobenzene)amido]-2,2-dimethyl-3,4-dihydro-2H-1-benzopyran-3-yl]oxy}phosphonic acid;
({[(3S,4S)-6-acetyl-4-[(4-fluorobenzene)amido]-2,2-dimethyl-3,4-dihydro-2H-1-benzopyran-3-yl]oxy}methoxy)phosphonic acid;
{[(3S,4S)-6-Acetyl-4-[(3-chlorobenzene)amido]-2,2-dimethyl-3,4-dihydro-2H-1-benzopyran-3-yl]oxy}phosphonic acid; and
({[(3S,4S)-6-acetyl-4-[(3-chlorobenzene)amido]-2,2-dimethyl-3,4-dihydro-2H-1-benzopyran-3-yl]oxy}methoxy)phosphonic acid.

2. A pharmaceutical composition comprising a compound, a hydrate, solvate, or a pharmaceutically acceptable salt thereof, the compound selected from:
(3S,4S)-6-Acetyl-4-[(3-chloro-4-fluorobenzene)amido]-2,2-dimethyl-3,4-dihydro-2H-1-benzopyran-3-yl (2S)-2-aminopropanoate,
(3S,4S)-6-Acetyl-4-[(3-chloro-4-fluorobenzene)amido]-2,2-dimethyl-3,4-dihydro-2H-1-benzopyran-3-yl (2S)-2-amino-3-methylbutanoate, and
(3S,4S)-6-Acetyl-4-[(3-chloro-4-fluorobenzene)amido]-2,2-dimethyl-3,4-dihydro-2H-1-benzopyran-3-yl (2S)-2-(methylamino)propanoate
together with one or more pharmaceutically acceptable carriers and/or excipients.

3-4. (canceled)

5. A method of treatment of a disease or medical condition, comprising administering to a subject suffering from such disease or condition an effective amount of a compound, a hydrate, a solvate, or a pharmaceutically acceptable salt thereof, the compound selected from:
(3S,4S)-6-Acetyl-4-[(3-chloro-4-fluorobenzene)amido]-2,2-dimethyl-3,4-dihydro-2H-1-benzopyran-3-yl (2S)-2-aminopropanoate,
(3S,4S)-6-Acetyl-4-[(3-chloro-4-fluorobenzene)amido]-2,2-dimethyl-3,4-dihydro-2H-1-benzopyran-3-yl (2S)-2-amino-3-methylbutanoate, and
(3S,4S)-6-Acetyl-4-[(3-chloro-4-fluorobenzene)amido]-2,2-dimethyl-3,4-dihydro-2H-1-benzopyran-3-yl (2S)-2-(methylamino)propanoate,
wherein the disease or condition is selected from migraine, aura with or without migraine, epilepsy, non-epileptic seizures, cerebrovascular accidents including stroke, intracranial haemorrhage (including or traumatic brain injury, epidural hematoma, subdural hematoma and subarachnoid haemorrhage), and intra-cerebral haemorrhage (including CADASIL), spinal cord vascular accidents arising from trauma, epidural hematoma, subdural hematoma or subarachnoid haemorrhage, pain including pain arising from hyperalgesia caused by damage to sensory neurons (i.e. neuropathic pain including but not limited to diabetic neuropathy, polyneuropathy, cancer pain, fibromyalgia, myofascial pain, post herpetic neuralgia, spinal stenosis, HIV pain, post-operative pain, post-trauma pain) or inflammation (including pain associated with osteoarthritis, rheumatoid arthritis, sciatica/radiculopathy, pancreatitis, tendonitis), neurodegenerative disease (including but not limited to Alzheimer's Disease, Parkinson's Disease, Huntington's Disease and Amyotrophic Lateral Sclerosis) and cardiovascular disease including myocardial infarction, coronary revascularization or angina.

6. A pharmaceutical composition according to claim 2 formulated as a liquid for intravenous dosage.

7. A pharmaceutical composition according to claim 2 formulated as a solid for oral dosage.

8. A pharmaceutical composition comprising a compound, a hydrate, a solvate, or a pharmaceutically acceptable salt thereof, the compound selected from:
(3S,4S)-6-Acetyl-4-[(3-chloro-4-fluorobenzene)amido]-2,2-dimethyl-3,4-dihydro-2H-1-benzopyran-3-yl (2R)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-4-methylpentanoate;
(3S,4S)-6-Acetyl-4-[(3-chloro-4-fluorobenzene)amido]-2,2-dimethyl-3,4-dihydro-2H-1-benzopyran-3-yl (2S)-pyrrolidine-2-carboxylate;
(3S,4S)-6-Acetyl-4-[(3-chloro-4-fluorobenzene)amido]-2,2-dimethyl-3,4-dihydro-2H-1-benzopyran-3-yl 2-amino-2-methylpropanoate;
(3S,4S)-6-Acetyl-4-[(3-chloro-4-fluorobenzene)amido]-2,2-dimethyl-3,4-dihydro-2H-1-benzopyran-3-yl 3-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2,6-diaminohexanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-carbamoylpropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-4-carbamoylbutanoate;
(3S,4S)-6-Acetyl-4-[(3-chloro-4-fluorobenzene)amido]-2,2-dimethyl-3,4-dihydro-2H-1-benzopyran-3-yl (2S)-2-amino-5-carbamimidamidopentanoate;
(3S)-4-{[(3S,4S)-6-Acetyl-4-[(3-chloro-4-fluorobenzene)amido]-2,2-dimethyl-3,4-dihydro-2H-1-benzopyran-3-yl]oxy}-3-amino-4-oxobutanoic acid;
(4S)-5-{[(3S,4S)-6-Acetyl-4-[(3-chloro-4-fluorobenzene)amido]-2,2-dimethyl-3,4-dihydro-2H-1-benzopyran-3-yl]oxy}-4-amino-5-oxopentanoic acid;
({[(3S,4S)-6-Acetyl-4-[(3-chloro-4-fluorobenzene)amido]-2,2-dimethyl-3,4-dihydro-2H-1-benzopyran-3-yl]oxy}methoxy)(methoxy)phosphinic acid;
N-[(3S,4S)-6-Acetyl-3-{[(2S)-2,3-dihydroxypropoxy]methoxy}-2,2-dimethyl-3,4-dihydro-2H-1-benzopyran-4-yl]-3-chloro-4-fluorobenzamide;
{[(3S,4S)-6-Acetyl-4-[(4-fluorobenzene)amido]-2,2-dimethyl-3,4-dihydro-2H-1-benzopyran-3-yl]oxy}phosphonic acid;
({[(3S,4S)-6-acetyl-4-[(4-fluorobenzene)amido]-2,2-dimethyl-3,4-dihydro-2H-1-benzopyran-3-yl]oxy}methoxy)phosphonic acid;
{[(3S,4S)-6-Acetyl-4-[(3-chlorobenzene)amido]-2,2-dimethyl-3,4-dihydro-2H-1-benzopyran-3-yl]oxy}phosphonic acid; and
({[(3S,4S)-6-acetyl-4-[(3-chlorobenzene)amido]-2,2-dimethyl-3,4-dihydro-2H-1-benzopyran-3-yl]oxy}methoxy)phosphonic acid
together with one or more pharmaceutically acceptable carriers and/or excipients.

9. A method of treatment of a disease or medical condition, comprising administering to a subject suffering from such disease or condition an effective amount of a compound, a hydrate, a solvate, or a pharmaceutically acceptable salt thereof, the compound selected from:
(3S,4S)-6-Acetyl-4-[(3-chloro-4-fluorobenzene)amido]-2,2-dimethyl-3,4-dihydro-2H-1-benzopyran-3-yl (2R)-2-amino-3-methylbutanoate;
(3S,4S)-6-Acetyl-4-[(3-chloro-4-fluorobenzene)amido]-2,2-dimethyl-3,4-dihydro-2H-1-benzopyran-3-yl (2R)-2-amino-4-methylpentanoate;
(3S,4S)-6-Acetyl-4-[(3-chloro-4-fluorobenzene)amido]-2,2-dimethyl-3,4-dihydro-2H-1-benzopyran-3-yl (2S)-pyrrolidine-2-carboxylate;
(3S,4S)-6-Acetyl-4-[(3-chloro-4-fluorobenzene)amido]-2,2-dimethyl-3,4-dihydro-2H-1-benzopyran-3-yl 2-amino-2-methylpropanoate;
(3S,4S)-6-Acetyl-4-[(3-chloro-4-fluorobenzene)amido]-2,2-dimethyl-3,4-dihydro-2H-1-benzopyran-3-yl 3-aminopropanoate;
(3S,4S)-6-Acetyl-4-[(3-chloro-4-fluorobenzene)amido]-2,2-dimethyl-3,4-dihydro-2H-1-benzopyran-3-yl (2S)-2,6-diaminohexanoate;
(3S,4S)-6-Acetyl-4-[(3-chloro-4-fluorobenzene)amido]-2,2-dimethyl-3,4-dihydro-2H-1-benzopyran-3-yl (2S)-2-amino-3-carbamoylpropanoate;
(3S,4S)-6-Acetyl-4-[(3-chloro-4-fluorobenzene)amido]-2,2-dimethyl-3,4-dihydro-2H-1-benzopyran-3-yl (2S)-2-amino-4-carbamoylbutanoate;
(3S,4S)-6-Acetyl-4-[(3-chloro-4-fluorobenzene)amido]-2,2-dimethyl-3,4-dihydro-2H-1-benzopyran-3-yl (2S)-2-amino-5-carbamimidamidopentanoate;
(3S)-4-{[(3S,4S)-6-Acetyl-4-[(3-chloro-4-fluorobenzene)amido]-2,2-dimethyl-3,4-dihydro-2H-1-benzopyran-3-yl]oxy}-3-amino-4-oxobutanoic acid;
(4S)-5-{[(3S,4S)-6-Acetyl-4-[(3-chloro-4-fluorobenzene)amido]-2,2-dimethyl-3,4-dihydro-2H-1-benzopyran-3-yl]oxy}-4-amino-5-oxopentanoic acid;
({[(3S,4S)-6-Acetyl-4-[(3-chloro-4-fluorobenzene)amido]-2,2-dimethyl-3,4-dihydro-2H-1-benzopyran-3-yl]oxy}methoxy)(methoxy)phosphinic acid;
N-[(3S,4S)-6-Acetyl-3-{[(2S)-2,3-dihydroxypropoxy]methoxy}-2,2-dimethyl-3,4-dihydro-2H-1-benzopyran-4-yl]-3-chloro-4-fluorobenzamide;
{[(3S,4S)-6-Acetyl-4-[(4-fluorobenzene)amido]-2,2-dimethyl-3,4-dihydro-2H-1-benzopyran-3-yl]oxy}phosphonic acid;
({[(3S,4S)-6-acetyl-4-[(4-fluorobenzene)amido]-2,2-dimethyl-3,4-dihydro-2H-1-benzopyran-3-yl]oxy}methoxy)phosphonic acid;
{[(3S,4S)-6-Acetyl-4-[(3-chlorobenzene)amido]-2,2-dimethyl-3,4-dihydro-2H-1-benzopyran-3-yl]oxy}phosphonic acid; and
({[(3S,4S)-6-acetyl-4-[(3-chlorobenzene)amido]-2,2-dimethyl-3,4-dihydro-2H-1-benzopyran-3-yl]oxy}methoxy)phosphonic acid,
wherein the disease or condition is selected from migraine, aura with or without migraine, epilepsy, non-epileptic seizures, cerebrovascular accidents including stroke, intracranial haemorrhage (including or traumatic brain injury, epidural hematoma, subdural hematoma and subarachnoid haemorrhage), and intra-cerebral haemorrhage (including CADASIL), spinal cord vascular accidents arising from trauma, epidural hematoma, subdural hematoma or subarachnoid haemorrhage, pain including pain arising from hyperalgesia caused by damage to sensory neurons (i.e. neuropathic pain including but not limited to diabetic neuropathy, polyneuropathy, cancer pain, fibromyalgia, myofascial pain, post herpetic neuralgia, spinal stenosis, HIV pain, post-operative pain, post-trauma pain) or inflammation (including pain associated with osteoarthritis, rheumatoid arthritis, sciatica/radiculopathy, pancreatitis, tendonitis), neurodegenerative disease (including but not limited to Alzheimer's Disease, Parkinson's Disease, Huntington's Disease and Amyotrophic Lateral Sclerosis) and cardiovascular disease including myocardial infarction, coronary revascularization or angina.

10. A pharmaceutical composition according to claim 8 formulated as a liquid for intravenous dosage.

11. A pharmaceutical composition according to claim 8 formulated as a solid for oral dosage.
